(19) United States
(12) Patent Application Publication
(10) Pub. No.: US 2021/0379243 A1
(43) Pub. Date: Dec. 9, 2021

(54) Artificial bionic blood vessel and preparation method

(71) Applicant: Haowei Ti, Xinxiang (CN)

(72) Inventors: Haowei Ti, Xinxiang (CN); Shiqi Mei, Shimen (CN)

(21) Appl. No.: 17/407,131

(22) Filed: Aug. 19, 2021

(51) Int. Cl. A61L 27/24; A61L 27/36; C09D 189/06; D06M 11/79; D03D 3/02; D03D 1/00; D03D 15/233

(52) U.S. Cl. CPC A61L 27/24; A61L 27/3604; C09D 189/06; D06M 11/79; D03D 3/02; D06M 2101/12; D03D 15/233; A61L 2430/22; A61L 2430/40; A61L 2420/02; D03D 1/00

(57) ABSTRACT

The present invention provides an artificial bionic blood vessel and a manufacturing method thereof. The artificial bionic blood vessel includes a three-layer-structured artificial bionic blood vessel body, where the three-layer structure of the artificial bionic blood vessel consists of a natural silk layer, a diluted liquid silica-gel layer and a weaved tube layer, the diluted liquid silica-gel layer is located on the inner side of the natural silk layer, the weaved tube layer is located on the outer side of the natural silk layer, and the weaved tube layer is made of catgut by weaving.

### TECHNICAL FIELD

The present invention belongs to the technical field of artificial blood vessel, and in particular, to an artificial bionic blood vessel and a manufacturing method thereof.

### BACKGROUND ART

In recent years, various cardiovascular diseases have seriously impacted human health, so blood vessel transplantation has attracted more and more attention. Autologous blood vessels are the best choice for blood vessel transplantation, however, due to its limited sources, autologous blood vessels cannot satisfy people's demands on blood vessel transplantation. In this case, the adoption of artificial blood vessels becomes a good choice.

But in the prior art, artificial blood vessels are mainly prepared from polytetrafluoroethylene or polyester-fiber fabrics, thus, the artificial blood vessels need to be sutured carefully during operations, and anastomotic stomas easily form stenosis and thrombus, especially for small diameter artificial blood vessels, the occurrence of stenosis and thrombus, is more probable; moreover, artificial blood vessels are easily broken when being bent, because the inner diameters of the artificial blood vessel are changed, and then blood flows are obstructed; and meanwhile, in actual use, conventional artificial blood vessels cannot be absorbed well by regenerated blood vessels, which further increases the occurrence of thrombus.

### SUMMARY OF THE PRESENT INVENTION

The purpose of the present invention is to provide an artificial bionic blood vessel and a manufacturing method thereof, which aims to solve the problems in the background art: in the prior art, artificial blood vessels are mainly prepared from polytetrafluoroethylene or polyester-fiber fabrics, thus, the artificial blood vessels need to be sutured carefully during operations, anastomotic stomas easily form stenosis and thrombus, and especially for small-diameter artificial blood vessels, the occurrence of stenosis and thrombus is more probable; and moreover, artificial blood vessels are easily broken when being bent, because the inner diameters of the artificial blood vessel are changed, and then blood flows are obstructed.

To solve the problems above, the present invention provides the following technical solution: an artificial bionic blood vessel includes a three-layer-structured artificial bionic blood vessel body, where the three-layer structure of the artificial bionic blood vessel consists of a natural silk layer, a diluted liquid silica gel layer and a weaved tube layer, the diluted liquid silica-gel layer is located on the inner side of the natural silk layer, the weaved tube layer is located on the outer side of the natural silk layer, and the weaved tube layer is made of catgut by weaving.

As a preferable technical solution of the present invention, the surface of the weaved tube layer is provided with a double spiral bulge.

As a preferable technical solution of the present invention, the ends of the natural silk layer, the diluted liquid silica-gel layer and the weaved tube layer are respectively provided with an elliptical bulge, and a ring-shaped junction of which the middle part is slightly bulging is formed through the three elliptical bulges.

As a preferable technical solution of the present invention, the ring-shaped junction is made of silicone grease or animal fat.

As a preferable technical solution of the present invention, the surface of the weaved tube layer and the surface of the ring-shaped junction are coated with fish-skin collagen oligopeptides subjected to liquification treatment.

A method for manufacturing an artificial bionic blood vessel includes steps of manufacturing the artificial bionic blood vessel as described in any one of the technical solutions above, and specifically includes the following steps.

S1, selecting natural silk with a diameter of 5-10 μm, and after carrying out sterilization and disinfection on the natural silk, weaving the obtained natural silk so as to form a natural silk layer;

S2, selecting and diluting liquid silica-gel so as to form a silica-gel solvent, and finally, solidifying the silica-gel solvent and then adsorbing the solidified solvent to the inner side of the natural silk layer so as to form a diluted liquid silica-gel layer;

S3, selecting and weaving catgut with a diameter of less than 40 μm so as to form a seamless tubular structure, and fixing the seamless tubular structure on the outer side of the natural silk layer so as to form a weaved tube layer; and

S4, selecting silicone grease or animal fat to prepare a ring-shaped junction.

As a preferable technical solution of the present invention, gaps in the natural silk layer (1) in S1 are filled with gel that can be absorbed by human bodies.

As a preferable technical solution of the present invention, the weaved tube layer (3) in S3 and the ring-shaped junction (4) in S4 are respectively coated with a vascular anastomosis adhesive when being applied to vascular connection.

Compared with the prior art, the present invention has the following beneficial effects:
1) the natural silk layer weaved by natural silk can form a protective layer to ensure that mutual repulsion will not be caused between a native ;regenerative blood vessel and the diluted liquid silica-gel layer, and ensure the integrity and smoothness of the inner wall of the diluted liquid silica-gel layer so as to avoid the occurrence of blockage; and moreover, the arrangement of the double spiral bulges can increase the toughness and elasticity of the artificial bionic blood vessel, thereby preventing the occurrence of adhesion of the inner wall after implantation and further preventing the occurrence of thrombus;
2) The artificial bionic blood vessel of the present invent ion mainly consists of animal tissues and natural biomaterials, thus, the artificial bionic blood vessel will not have side effects on body functions and is easily absorbed by human bodies; a fish-skin collagen oligopeptides coating is distributed on a junction between the bionic blood vessel and a blood vessel, so that the regeneration function of the blood vessel is prompted; and the outermost layer is in the shape of an ultra-fine catgut weaved tube, and after a period of time, the ultra-fine catgut weaved tube will be absorbed and covered by a regenerative blood vessel;
3) A ring-shaped junction made of silicone grease or animal fat is disposed at the junction of the artificial bionic blood vessel of the present invention, thus, the artificial bionic blood vessel can be directly absorbed to a native blood vessel, thereby avoiding suturing as much as possible; and
4) The fish-skin collagen oligopeptides subjected to liquification treatment can be completely absorbed by the catgut, and provide a nutrient source when a native blood vessel regenerates, thereby promoting the growth of the native blood vessel.

### BRIEF DESCRIPTION OF THE DRAWINGS

The accompanying drawings are adopted to further illustrate the present invention, constitute a part of the description, and explain the present invention with reference to the embodiments, but not limit the present invention. In the drawings:

FIG. 1 is a structural schematic diagram of the present invention;

FIG. 2 is a structural schematic diagram of a tube body structure of the present invention;

FIG. 3 is a sectional view of FIG. 2 in the present invention;

FIG. 4 is a structural schematic diagram of the bulge of the present invention; and

FIG. 5 is a structural schematic diagram of an embodiment 2 of the present invention

FIG. 6 is an electron micrograph of the artificial bionic blood vessel of the present invention 18 months after being transplanted in a living body;

FIG. 7 is a cross-sectional view of the artificial bionic blood vessel of the present invention 18 months after being transplanted in a living body.

In the figures: 1, natural silk layer; 2, diluted liquid silica-gel layer; 3, weaved tube layer; 31, bulge; 4, ring-shaped junction; 5, petaloid junction.

### DETAILED DESCRIPTION OF EMBODIMENTS

The following clearly and completely describes the technical solutions in the embodiments of the present invention with reference to the accompanying drawings in the embodiments of the present invention. It is obvious that the described embodiments are only a part rather than all of the embodiments of the present invention. All other embodiments obtained by persons of ordinary skill in the art based on the embodiment of the present invention without creative efforts shall fall within the protection scope of the present invention.

### Embodiment 1

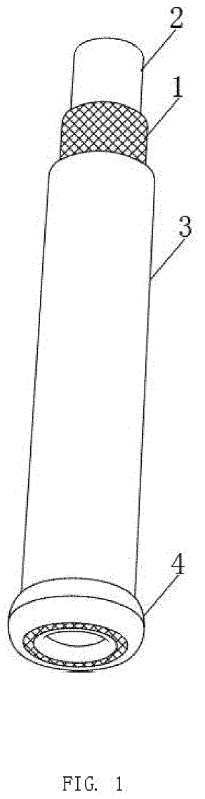

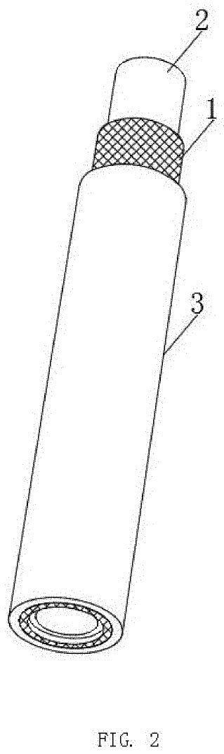

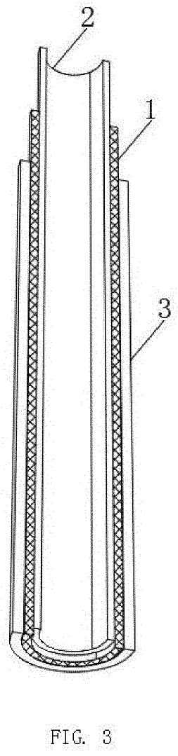

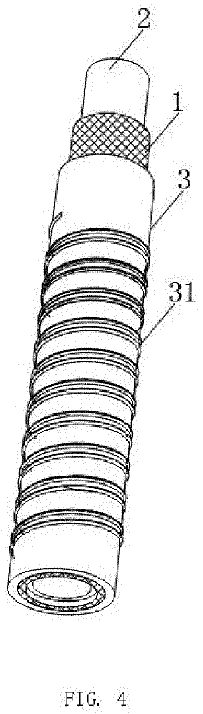

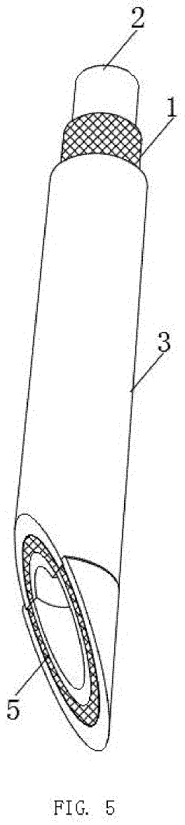

As shown in FIG. 1-4, the present invention provides the following technical solution: an artificial bionic blood vessel includes ,a three-layer-structured artificial bionic blood vessel body, where the three-layer structure of the artificial bionic blood vessel consists of a natural silk layer 1, a diluted liquid silica-gel layer 2 and a weaved tube layer 3, the diluted liquid silica-gel layer 2 is located on the inner side of the natural silk layer 1, the weaved tube layer 3 is located on the outer side of the natural silk layer 1, and the weaved tube layer 3 is made of catgut by weaving.

In this embodiment: the surface of the weaved tube layer 3 is provided with a double spiral bulge 31, and the double spiral bulge 31 can increase the overall toughness and elasticity of the bionic blood vessel body, so that the bionic blood vessel has better restorability when being applied to the insertion of blood vessels at joints, thereby avoiding the blockage of the blood vessel caused by the adhesion of the inner tube wall after the blood vessel is under pressure; and definitely, the bulge 31 also may be strip-shaped or ring-shaped and the like according to actual use needs.

In this embodiment: the ends of the natural silk layer 1, the diluted liquid silica-gel layer 2 and the weaved tube layer 3 are respectively provided with an elliptical bulge, a ring-shaped junction 4 of which the middle part is slightly bulging is formed through the three elliptical bulges, the ring-shaped junction 4 is made of silicone grease or animal fat and consists of animal tissues and natural biomaterials, thus, the artificial bionic blood vessel will not have side effects on body functions and is easily absorbed by human bodies; and raw materials are easy to obtain, which can reduce the manufacturing cost of the artificial bionic blood vessel to a certain extent.

In this embodiment: the surface of the weaved tube layer 3 and the surface of the ring-shaped junction 4 are both coated with fish skim collagen oligopeptides subjected to liquification treatment, and the fish-skin collagen oligopeptides subjected to liquification treatment can be completely absorbed by catgut, and provide a nutritional resource when a native blood vessel regenerates, so that the native blood vessel can grow better.

### Embodiment 2

As shown in ATG. 5, the ends of the natural silk layer 1, the dilute liquid silica-gel layer 2 and the weaved tube layer 3 are provided with petaloid lamellas, and multiple petaloid lamellas form a petaloid junction 5, so that the artificial bionic vessel may be connected to a native blood vessel better, thereby reducing the risks of endothelial hyperplasia and stenosis of anastomotic stomas.

A method for manufacturing an artificial bionic vessel includes, steps of manufacturing any artificial bionic vessel as described above, and specifically includes the following steps:

S1, selecting natural silk with a diameter of 5-10 μm, and after carrying out sterilization and disinfection on the natural silk, weaving the obtained natural silk so as to form a natural silk layer 1, where natural mulberry silk is selected and rolled into a tube by using a weaving technology, and then silk with different properties is woven outside the tube as a protective layer to prevent blood leakage, so that the diameter of the vessel may reach 1 mm, and no thrombus is easily caused; and the protein compositions of the silk can be compatible with tissues in the body, so the blood vessel is suitable for young patients whose blood vessels need to be replaced; gaps in the natural silk layer 1 are filled with get that can be absorbed by the human body, thus, the occurrence of blood outleakage when the artificial bionic blood vessel is inserted into the human body is further prevented, and compared with artificial compounds such as polyester fibers and the like, a finally prepared tube is more flexible, and the flexibility of the tube is more similar to that of native vessels;

S2, selecting and diluting liquid silica-gel so as to form a silica-gel solvent and finally, solidifying the silica-gel solvent and adsorbing the solidified solvent to the inner side of natural silk layer 1 so as to form a diluted liquid silica-gel layer 2, where because the liquid silica-gel prepared by taking high-purity two-component room-temperature silica gel as the basic raw material through catalyzing by platinum or peroxides is adopted, the liquid silica-gel layer is non-toxic and odorless, physiologically inert, resistant to biological aging, and has little reaction to human tissues, thus, the artificial bionic blood vessel will not cause a foreign body reaction after being implanted into human tissues, and cause no inflammation to surrounding tissues; and because the diluted liquid silica -gel layer 2 is taken as the outermost layer of the artificial bionic blood vessel, the flexibility of the artificial bionic blood vessel can be further increased

S3, selecting and weaving catgut with a diameter of less than 40 μm so as to form a seamless tubular structure, and fixing the seamless tubular structure on the outer side of the natural silk layer 1 so as to form a weaved tube layer 3, where after the artificial bionic vessel is connected to a native bionic vessel for a period of time, the catgut-weaved tube will be absorbed and covered by a regenerated vessel, thereby improving the possibility of success of vascular implantation treatment; and

S4, selecting silicone grease or animal fat to prepare a ring-shaped junction 4, where the weaved tube layer 3 and the ring-shaped junction 4 in S4 are respectively coated with a vascular anastomosis adhesive when being applied to vascular connection, and the silicone grease or animal fat can be directly adsorbed to a blood vessel, thereby avoiding suturing, as much as possible.

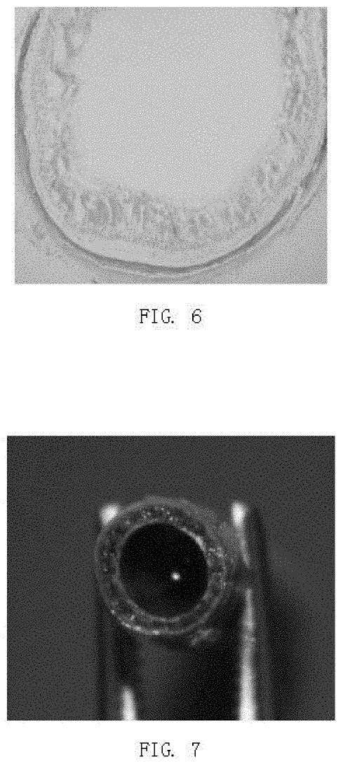

Specifically, as shown in FIG. 6 and FIG. 7, 18 months later after the artificial bionic vessel of the present invention is applied to in vivo transplantation, the inner wall is smooth, and the weaved tube layer 3 on the outer wall has been gradually absorbed and covered by the regenerated vessel, therefore, the artificial bionic vessel of the present invent ion has a huge market in clinical applications.

Finally, it should be noted that the foregoing embodiments are only preferable embodiments of the present invention and not intended to limit the present invention, although the present invention is described in detail with reference to the foregoing embodiments, a person of ordinary skill in the art still may modify the technical solutions recorded in the foregoing embodiments, or make equivalent substitutions for some of the technical features, and any modification, equivalent replacement, and improvement made without departing from the spirit and principle of the present invention shall fall within the protection scope of the present invention.

1. An artificial bionic blood vessel, comprising three-layer-structured artificial bionic blood vessel body, where the three-layer structure of the artificial bionic blood vessel consists of a natural silk layer (1), a diluted silica-gel layer (2) and a weaved tube layer (3), the diluted liquid silica-gel layer (2) is located on the inner side of the natural silk layer (1), the weaved tube layer (3) is located on the outer side of the natural silk layer (1), and the weaved tube layer (3) is made of catgut by weaving.

2. The artificial bionic blood vessel according to claim 1, wherein the surface of the weaved tube layer (3) is provided with a double spiral bulge (31).

3. The artificial bionic blood vessel according to claim 1, wherein the ends of the natural silk layer (1), the diluted liquid silica-gel layer (2) and the weaved tube layer (3) are respectively provided with an elliptical bulge, and a ring-shaped junction (4) of which the middle part is slightly bulging is formed through the three elliptical bulges.

4. The artificial bionic blood vessel according to claim 3, wherein the ring-shaped junction (4) is made of silicone grease or animal fat.

5. The artificial bionic blood vessel according to claim 1, wherein the surface of the weaved tube layer (3) and the surface of the ring-shaped junction (4) are both coated with fish-skin collagen oligopeptides subjected to liquification treatment.

6. A method for manufacturing an artificial bionic blood vessel, comprising steps of manufacturing the artificial bionic blood vessel as described in any one of claims 1-5, wherein the method specifically comprises the following steps:
S1, selecting natural silk with a diameter of 5-10 μm, and after carrying out sterilization and disinfection on the natural silk, weaving the obtained natural silk so as to form a natural silk layer (1);
S2, selecting and diluting liquid silica-gel so as to form a silica-gel solvent, and eventually, solidifying the silica-gel solvent and absorbing the solvent to the inner side of the natural silk layer (1) so as to form a diluted liquid silica-gel layer (2);
S3, selecting and weaving catgut with a diameter of less than 40 μm so as to form a seamless tubular structure, and fixing the seamless tubular structure on the outer side of the natural silk layer (1) so as to form a weaved tube layer (3); and
S4, selecting silicone grease or animal fat to prepare a ring-shaped junction (4).

7. The method for manufacturing an artificial bionic blood vessel according to claim 6, wherein gaps in the natural silk layer (1) in S1 are filled with gel that can be absorbed by human bodies.

8. The method for manufacturing an artificial bionic blood vessel according to claim 6, wherein the weaved tube layer (3) in S3 and the ring-shaped junction (4) in S4 are respectively coated with a vascular anastomosis adhesive when being applied to vascular connection.
